(19) United States
(12) Patent Application Publication
(10) Pub. No.: US 2007/0287147 A1
(43) Pub. Date: Dec. 13, 2007

(54) Method of Monitoring a Microorganism That Causes Infectious Disease of a Laboratory Animal

(75) Inventors: Teruyuki Nagamune, Saitama (JP); Akihiko Tajima, Chiba (JP); Yutaka Yamagata, Saitama (JP); Hiroyoshi Aoki, Tokyo (JP)

Correspondence Address: GREENBERG TRAURIG, LLP (SV);IP DOCKETING, 2450 COLORADO AVENUE, SUITE 400E, SANTA MONICA, CA, 90404, US

(73) Assignee: NAGAMUNE, TERUYUKI, SAITAMA JAPAN (JP)

(21) Appl. No.: 10/599,367

(22) PCT Filed: Mar. 2, 2005
(86) PCT No.: PCT/JP05/03526

§ 371 Date: Apr. 16, 2007

(30) Foreign Application Priority Data
Mar. 29, 2004 (JP) 2004-096271

(51) Int. Cl. C12Q 1/70; G01N 33/53

(52) U.S. Cl. 435/5; 435/7.21

(57) ABSTRACT

This invention provides a method to monitor a microorganism that causes infectious disease of a laboratory animal by using a micro flow channel chip immobilized with a molecular to be tested such as an antigen or an antibody of the microorganism that causes infectious disease, the method comprises flowing serum or body fluid taken from the laboratory animal through the minute flow channel of the micro flow channel chip and detecting the antigen antibody reaction on the chip. The method of this invention enabled medical inspection of an infectious disease of a laboratory animal and microorganism monitoring of a laboratory animal, by using minute amount of animal serum or body fluid in a closed system rapidly and sensitively.

### BACKGROUND OF THE INVENTION

1. Field of the Invention

This invention relates to a method of monitoring a microorganism that causes infectious disease of a laboratory animal using a micro flow channel chip. According to the method of the invention, pathogenic microorganism that causes infectious disease of a laboratory animal can be detected quickly and sensitively in a closed system, using a trace amount of animal serum or body fluid.

2. Related Art

When an experimenter is conducting experiments by handling animals, there is a danger that a pathogen harmful for human may be lurking in the experimental animals and the experimenter may be infected by the pathogen. It is possible that an experimental animal may die except for experimental handling because of the pathogen existing in the experimental animal. Moreover, it is possible that an experimental animal may be in the latency period of a pathogenic disease. In these eases, the reliability of the animal experiment is not assured and it may result in failure of the experiment. Considering such risk, there is a need to monitor a microorganism that causes infectious disease to laboratory animals. Moreover, by monitoring such microorganisms, the infection of the experimental animal may be found at an early stage, and the infectious microorganism may be identified. As a result, the degree of the infection can be assessed rapidly and accurately as possible, and some proper measure for safety and facility can be taken.

In the past, the enzyme-linked immunosorbent assay (ELISA method) has been widely used to monitor microorganisms that cause infectious disease to a laboratory animal. In the ELISA method, samples are prepared by diluting (normally diluted to 1/10˜1/50) a certain amount (normally about 100 μl) of blood taken from a laboratory animal, antigen antibody reaction is conducted between the sample and the antigen of the microorganism immobilized onto a 96 wells plate, and the infection is judged by detecting the antibody bound to the antigen by a secondary antibody labeled by enzymes or the like. The ELISA method is widely used in this technical field, and described in various textbooks, laboratory protocols and the like. For example, Manuals for handling infectious diseases of the laboratory animals: edited by Kazuyoshi Maejima, published by Adthree, Co Ltd. 2001, can be refereed.

### SUMMARY OF THE INVENTION

According to conventional methods used to monitor an infectious disease of a laboratory animal, massive blood is needed for one test (in the case of mouse, only 100 μl corresponds to 1/10 of total blood of the individual), therefore, there are some problems to be solved as follows,
(1) The microbiological condition of a parent population has to be estimated from the result of a random inspection of the parent population, and no other method can not be adopted.
(2) It is necessary to estimate the course of infection from plural number of different animal individuals, because a repetitive and continuous test on an identical individual can not be conducted.
(3) It take a long time for the test procedure and antigen antibody reaction.
(4) Instruments and careful attention to prevent infection of human is needed, because the testing and detecting procedures are carried out in an open system.

To resolve the problems described above, the invention provides a method to monitor a microorganism that causes infectious disease of a laboratory animal, which comprises immobilizing an antigen or an antibody of a microorganism that causes infectious disease of a laboratory animal onto a micro flow channel chip directly or indirectly, flowing a test sample from the laboratory animal through micro flow channel of the micro flow channel chip, conducting an antigen antibody reaction on the micro flow channel chip, and further detecting the antigen antibody reaction.

In addition, this invention provides a method to use a micro flow channel chip on which an antigen or an antibody of a microorganism that causes infectious diseases of a laboratory animal is directly or indirectly immobilized to monitor the microorganism.

Moreover, the invention provides a micro flow channel chip on which an antigen or an antibody of a microorganism that causes infectious diseases of a laboratory animal is directly or indirectly immobilized, and used to monitor the microorganism.

The present invention enabled to detect the infection of an animal by a microorganism efficiently and sensitivity, by conducting an antigen antibody reaction on a minute flow channel using a micro flow channel chip. According to the method of the invention, a test can be conducted by small amount of animal serum or body fluid (1/100 volume of the ordinal procedure), therefore, advantageous effects as follows can be obtained.
(1) The procedure from collecting blood or sample to conducting test operation is easy and simple.
(2) As to small animals such as mouse, rat and the like, burden to such animals is not heavy, therefore, frequent blood collection can be done on one animal individual, and a continuous test can be conducted.
(3) Monitoring of a microorganism can be conducted while proceeding experimental procedures.
(4) A population can be tested with high throughput.

In short, the method of this invention using a micro flow channel chip is conducted in a completely closed system, and the operation using this system can be conducted easily and rapidly. Therefore, it is also advantageous in that the risk of infection to a human body by an infectious microorganism and contamination of facility is low, thus a microorganism that causes infectious disease of a laboratory animal can be monitored in safety.

### BRIEF DESCRIPTION OF THE DRAWINGS

FIG. 1 is a figure showing the structure of the micro flow channel chip.

FIG. 2 is a photograph and a graph showing the result of detecting a reaction between mycoplasma antigen and its antibody on the micro flow channel chip.

FIG. 3 is a graph showing a correlation between dilution ratio of the antibody and the intensity of fluorescence.

FIG. 4 is a photograph showing the result of cross reaction test conducted on the micro flow channel chip.

### BEST MODE FOR CARRYING OUT THE INVENTION

This invention relates to a method to monitor a microorganism that causes infectious disease of a laboratory animal, which comprises immobilizing a molecular to be detected such as an antigen or an antibody of a microorganism that causes infectious disease of a laboratory animal onto a micro flow channel chip, flowing serum or body fluid obtained from the laboratory animal through micro flow channel of the micro flow channel chip, and detecting the antigen antibody reaction on the chip.

The inventors developed a micro flow channel chip for the purpose to provide a biomolecule microchip, which has a structure that enables to detect binding of various proteins or DNAs to other compounds on the microchip, to harvest the bound compounds, and to identify them. It was reported in Japanese application No. 2002-243734. In this specification, a micro flow channel chip means that described in Japanese application No. 2002-243734 or an altered micro flow channel chip according to the sample to be tested and the experimental conditions as needed. The micro flow channel chip used in this invention, however, should not be understood to be limited to that described in Japanese application No. 2002-243734, and other microchips can be also used within the spirit of the invention.

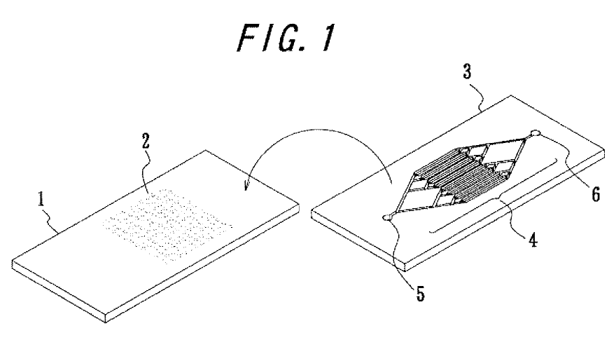

The micro flow channel chip described in Japanese application No. 2002-243734 is composed of spots of immobilized biomolecules, a substrate part that supports the spots, a minute flow channel part that supplies fluid, and a minute flow channel part for collecting the reactants. Therefore, using the micro flow channel chip described in Japanese application No. 2002-243734, binding between minute amount of biomolecule and the sample can be detected on the microchip, and the bound compound can be harvested for identified. FIG. 1 shows the structure of the micro flow channel chip described in Japanese application No. 2002-243734.

In the micro flow channel chip described in Japanese application No. 2002-243734 (FIG. 1), an array of biomolecules spots 2 (in the case of present invention, an antigen or an antibody of a pathogenic microorganism) are formed on the first substrate 1 made of glass or plastics. When biomolecules are immobilized on the substrate 1, the spots may be arranged to an array (FIG. 1). Otherwise, a straight or curved strip, or one having an arbitrary shape may be used instead of the spots. Such spots or strip may be formed to have an arbitrary angle and an arbitrary position toward the micro flow channel, by forming deposition using electrospray deposition method in accordance to the purpose of usage. The micro flow channel chip described in Japanese application No. 2002-243734 further has a second substrate 3, and the second substrate 3 has a concave portion 4 in one surface thereof. The one surface, having the concave potion 4 of the second substrate 3 is bonded to a surface, having spots 2, of the first substrate 1. Owing to the bonding, closed micro flow channels and reaction regions are built between the substrates or in a gap therebetween. Liquid to react with is then to be properly supplied to them.

Both ends of the concave potion 4 of the second substrate 3 have through holes respectively, which holes are used as an inlet 5 for supplying liquid and an outlet 6 for recovering the liquid, respectively. The microchip is designed such that the liquid poured into the inlet 5 is supplied to the micro supply flow channels, in which one flow channel is diverged into a number of channels, to uniformly be fed to all spots in parallel. In addition in the microchip, after the branched liquid passes through the spots it would be collected into one flow recovery channel along with confluence of the channels to be recovered from the outlet 6.

By conducting antigen antibody reaction on the minute flow channel of the micro flow channel chip having such structure, efficiency of the antigen antibody reaction can be improved, therefore, it enables detection of an antigen of a microorganism that causes infectious disease with high sensitivity and accuracy in a short period. According to the method of this invention using the micro flow channel chip, a microorganism that causes infectious disease can be monitored rapidly with high sensitively, by only collecting trace amount (less then 1/100 volume of conventional methods, 0.5-20 μl) of serum or body fluid from an experimental animal. Moreover, because the system of the micro flow channel chip is a completely closed detection system, the method of present invention is also advantageous in the aspect of safety. In addition, for the test can be conducted rapidly and simply using a minute volume sample, a repetitive and continuous monitoring of a microorganism is possible using a single individual.

According to the method of this invention, an antigen from a microorganism that causes infectious disease to a laboratory animal is spotted and immobilized on the substrate of the micro flow channel chip. Meanwhile, the word “antigen” used herein includes antigenic proteins, lipids, cell wall polysaccharides and the like from pathogenic microorganisms. As to the method for immobilization of the antigen, electrospray deposition method can be preferably adopted, however, it is not limited to it. Electrospray deposition method is well-known to those skilled in the art, and the description in international publication No. WO98/58745 can be used as a reference, for example.

The surface of the immobilized substrate may preferably be coated with a functional group such as aldehyde, epoxy, succinimide, malcimide, thiol, amino, carbonyl and the like. However, the functional groups used to coat the surface are not limited to them.

In addition, after immobilization of the antigenic protein and the like, it is preferable to conduct a blocking reaction using a protein solution such as skim milk or bovine serum albumin, in order to prevent non-specific adsorption of the proteins contained in the test sample that is passed through afterwards.

The test sample obtained from the laboratory animal to be tested are passed through the flow channel of the micro flow channel chip on which an antigen of a pathogenic microorganism is immobilized, and then they can be reacted on the micro flow channel chip. If antibody toward the antigen is present in the test sample, the antibody reacts with the immobilized antigen. A period for the antigen antibody reaction is not particularly limited, it may preferably be from about 5 min to 30 min. Then after the reaction, buffer solution may be passed through the channel to rinse out the antibodies not bound to the antigen.

Afterwards, a labeled secondary antibody that can recognize above-mentioned antibody may be passed through, thus antibody bound to antigen may be detected by the labeling of secondary antibody. For example, in the case of detecting an antibody derived from mouse, the antibody bound to the antigen immobilized on the micro flow channel chip can be detected using anti-mouse antibody labeled with fluorescence using as a secondary antibody, for example. The means to label antibodies is not limited to fluorescence label, radiolabeled antibodies or antibodies labeled with an enzyme (such as horseradish peroxidase, alkaline phosphatase) that binds to secondary antibodies can be also used. When an animal to be tested is infected or has an infectious record by the pathogenic microorganism from which the antigen is derived, the infection or infectious history can be determined from the amount of the antibody bound to the antigen, because the antibody against the antigen is present in serum of the animal.

Antibody can be immobilized on the substrate of the micro flow channel chip, using electrospray deposition method. In such case, it is assumed that the immobilized antibody reacts the antigen existing in the test sample. The antigen in the test sample can be also detected by adopting such method, and such embodiment is also within the scope of this invention. Although the infectious history cannot be detected by the means to detect an antigen, this method is applicable in the case the pathogenic microorganism infected is present in the test animal.

By the way, the method described above corresponds the embodiment where an antigen or an antibody is directly immobilized on the micro flow channel chip. However, as described below, an antigen or an antibody may be indirectly immobilized on the substrate of the micro flow channel chip using a ligand that specifically recognize a tag attached to the antigen or the antibody, and such embodiment is also within the scope of this invention. Here, as the examples of the ligand that specifically recognize the tag attached to an antigen or an antibody, avidin, glutathione and nickel chelate group can be listed, moreover, amylase and anti-tag antibodies such as anti-FLAG antibody can be also listed. However, it is not limited to these ligands, other ligands can be also used ad libitum. For example, avidin is a ligand that specifically recognizes biotin, therefore, protein attached with biotin tag is assumed to bind to the surface of substrate immobilized with avidin.

E. coli or cells expressing an antigen or an antibody of a pathogenic microorganism introduced with biotin tag, glutathione-S-transferase tag, histidine tag, maltose binding protein tag or FLAG tag can be produced by genetic recombination. When crude extract solution derived from E. coli or cells described above, or protein purified from them was passed through the micro flow channel chip on which the specific ligand described above is immobilized, the specific ligand described above and the antigen contained in the crude extract binds via the tag as described above. That is, an antigen can be immobilized indirectly on the substrate of the micro flow channel chip via the ligand. After indirect immobilization of the antigen of the pathogenic microorganism, the test sample can be passed through the channel of the micro flow channel chip, antigen antibody reaction can be conducted on the micro flow channel chip, and the antibody in the test sample can be detected.

Moreover, as to alternative methods for indirect immobilization, a secondary antibody toward an antibody that recognize an antigen (i.e. a primary antibody) can be immobilized on the substrate of the micro flow channel chip. Then by flowing an antigen or a primary antibody through the micro flow channel, the antigen or the primary antibody can bind on the substrate. As described above, the antigen or the primary antibody can also indirectly bind on the substrate of the micro flow channel chip via the secondary antibody. Moreover, by conducting antigen antibody reaction on the flow channel of the micro flow channel chip by flowing the test sample through the flow channel of the micro flow channel chip, the antigen or the antibody in the test sample can be detected.

The method to immobilize an antigen or an antibody is not limited to the embodiment where the antigen or the antibody is immobilized on the substrate of the micro flow channel chip. As an alternative embodiment, an antigen or an antibody to be immobilized can be immobilized on the surface of microbeads or nanofibers, such microbeads can be inserting into the flow channel, and the identical effect can be obtained.

A barrier can be placed in the midway of the flow channel, microbeads or nanofibers on which an antigen or an antibody is immobilized can be passed through, then the microbeads or nanofibers are banked up by the barrier. Afterwards, the test samples can be passed through flow channel of the micro flow channel chip, then antigen antibody reaction occur between the antigen immobilized on the microbeads or nanofibers and the antibody in the test sample, thereby antibody in the test sample can be detected.

Moreover, microbeads or nanofibers immobilized with a ligand that specifically binds to an antigen or a secondary antibody can be used to immobilize an antigen on the microbeads or the nanofibers. In concrete, microbeads or nanofibers can be bound with a ligand that specifically recognize a tag attached to an antigen or with a secondary antibody, and such microbeads or nanofibers can be passed through the micro flow channel with a barrier placed in the midway of the flow channel, thereby the microbeads or nanofibers are banked up in the middle of the flow channel.

Afterwards, an antigen attached with a tag can be produced by the technique of genetic recombination or a native antigen can be passed through the flow channel, then the antigen specifically binds to the banked up microbeads or nanofibers via the tag or the secondary antibody on the microbeads or nanofibers, thereby, the antigen can be immobilized indirectly on the microbeads or nanofibers. Then, the test sample can be passed through the flow channel in which the microbeads or nanofibers indirectly immobilized with the antigen are in existence, thereby the presence of the antibody in the test sample can be detected.

The size of the microbeads or the nanofibers used herein may preferably be in the range of a several μm to several dozen μm. The material of the microbeads or the nanofibers may be polysaccadrides such as agarose, dextran, cellulose, chitosan, and synthetic polymers such as polyacrylamide, polystyrene, polyvinyl alcohol, polyethylene glycol. However, the size and the material of the microbeads or the nanofibers are not limited within such range, and a skilled artisan can select an appropriate one properly ad libitum.

The surface of the microbeads or the nanofibers may preferably be coated with a functional group such as aldehyde, epoxy, succinimide, maleimide, thiol, amino, carbonyl and the like. However, the functional group that coats the surface is not limited to them.

As a specific example for the microbeads, latex beads made of polystyrene (Sigma, average particle size of 0.1 μm, 0.3 μm, 0.46 μm, 0.6 μm) can be listed. For the surface of the beads is hydrophobic, they can absorb proteins, thus used for immobilization of an antigen, an antibody or a ligand.

As a test sample according to this invention, serum, plasma, urine, lymph fluid, and spinal fluid derived from the laboratory animal to be tested can be listed, and serum and plasma are particularly preferred. However, the body fluid to be used as the test sample is not limited to them, and a skilled artisan can properly select various samples as needed ad libitum.

As a laboratory animal which is the subject to monitor microorganism that causes infectious disease in this invention, mouse, rat, guinea pig, hamster, rabbit, cat, pig, monkey, bird, dog and the like can be listed, and mouse and rat are the most commonly used as a laboratory animal. However, the animal is not limited to the examples described above, other animals used as a laboratory animal can be also monitored for a pathogenic microorganism by the method according to this invention. In addition, in these days, transgenic animals (genetically modified laboratory animals) have been widely used in the research of biochemistry and medical science, such transgenic animals can be also monitored for a pathogenic microorganism by the method according to this invention.

As microorganisms to be monitored in this invention, microorganisms described in Manuals for handling infectious diseases of the laboratory animals, edited by Kazuyoshi Maejima, published by Adthree, Co Ltd. 2001, page 21, literature 1-2 can be listed. However, the range of the microorganisms to be detected by the method of this invention is not particularly limited, various microorganisms can be monitored. Therefore, microorganisms to be monitored are not limited to the microorganisms described in the above-mentioned literature.

Moreover, in the case that the laboratory animal is mouse, the main microorganisms to be subjected to medical inspection or monitoring are as follows; Mouse hepatitis virus (MHV), Sendai virus (HVJ), Ectomrlia virus, Mouse adenovirus, Lymphocytic choriomeningitis virus (LCMV), Hantaan virus, Mycoplasma pulmonis, Clostridium piliforme, Pneumonia virus of Mice, Mouse rotavirus (EDIMV), Mouse parvovirus (MVM/MPV), Mouse encephalomyelitis virus (TMEV), Pneumonia virus of Mice (PVM), Mouse Adenovirus, Reovirus type 3, lactose dehydrogenase elevating virus, Clostridium piliforme, Corynebacterium kutscheri, Pasteurella pneumotropica, Cilia-associated respiratory (CAR) bacillus, Escherichia coli O115 a,c;K(B), Helicobactor hepaticus, Psudomonas aeruginosa, Staphylococcus aureus, Pneumocystis carinii, Giardia muris, Spironucleus muris, and Helminths (pinworms).

Moreover in the case that the laboratory animal is rat, the main microorganisms to be subjected to medical inspection or monitoring are as follows; Mouse hepatitis virus (MHV), Sendai virus (HVJ), Mouse adenovirus, Hantaan virus, Mycoplasma pulmonis, Clostridium piliforme, Pneumonia virus of Mice, Rat parvovirus (KRV/H-1/RPV), Mouse encephalomyelitis virus (TMEV), Pneumonia virus of Mice (PVM), Mouse Adenovirus, Reovirus type 3, Clostridium piliforme, Corynebacterium kutscheri, Bordetella bronchiseptia, Pasteurella pneumotropica, Streptococcus pneumoniae, Cilia-associated respiratory (CAR) bacillus, Psudomonas aeruginosa, Staphylococcus aureus, Pneumocystis carinii, Giardia muris, Spironucleus muris, and Helminths (pinworms).

### EXAMPLES

The following examples and figures are intended to further illustrate the invention, however, the descriptions are not to limit the range of this invention in any way.

### Example 1

In the following experiments, PBS (Na₂HPO₄ 0.61 g, KH₂PO₄ 0.19 g, NaCl 8.00 g, KCl 0.20 g, MilliQ (Millipore) IL), PBST (0.05% Tween20-PBS), and a washing solution (2% skim milk-PBST), and a blocking solution (2% skim milk-PBST) were used, and they were composed of the compositions described in brackets. Surface of the substrate, where antigen antibody reaction was conducted, was coated with Indium-Tin Oxide (ITO) and a glass substrate (manufactured by Tatsunami glass, 26×76 mm) introduced with aldehyde group was used on the coated substrate.

At first, about 0.45 μg of mycoplasma (MP) antigen (DENKA SEIKEN Co., Ltd.) was sprayed on the substrate using an electrospray deposition device (manufactured by Fuence). At conducting the spray, a glass mask having a slit (width 200 μm, length 12 mm) was used. By using the mask, antigens were deposited on the substrate in the shape of thin linear figure. The substrate was set with a flow channel made of polydimethylsiloxane having 8 slits (width 400 μm, depth 100 μm), in which the sprayed side was faced to the flow channel side. The flow channel extended along with the long axis of the substrate, while the antigen extended along with the short axis of the substrate. Therefore, antigen antibody reaction occurred at the point where the both axis crossed, the existences of antibody against a specific pathogenic microorganism could be detected as box-shaped spots.

Next, a reaction was conducted at the flow channel for 10 min at 30° C. under the condition of saturated water vapor, thereby cross-linking reaction between the aldehyde group on the substrate and the antigen protein was achieved. Then 3 μl of the washing solution was passed through 3 times for each flow channels, and unreacted antibodies were rinse out. Next 3 μl of the blocking solution was applied to conduct a blocking reaction for 10 min at room temperature.

After the blocking reaction, anti-mycoplasma antibody derived from mouse (DENKA SEIKEN Co., Ltd.) was diluted sequentially with MilliQ water (MILLIPORE Co., Ltd.), and 3 μl of the diluted solution was passed through each flow channels. Thereafter, the antigen antibody reaction was conducted for 10 min at room temperature. Then the flow channels were washed with 3 μl of PBST for 3 times to rinse out the non-bonded and excess antibodies.

Thereafter, 10 μg/ml of anti-mouse antibody labeled with Alexa Fluor 488 (Molecular Probes Co., Ltd.) in the blocking solution was passed through as the second antibody in increments of 3 μl, and the antigen antibody reaction was conducted for 10 min at room temperature. Thereafter, they were washed 3 times with 3 μl of PBST and PBS respectively, and excessive labeled antibodies were rinsed out.

Fluorescence of Alexa488 was measured by OLYMPUS SRX9 microscope equipped with a cooled CCD camera. Intensities of the fluorescence per spots were determined from the measured images using ArrayPro (planetron).

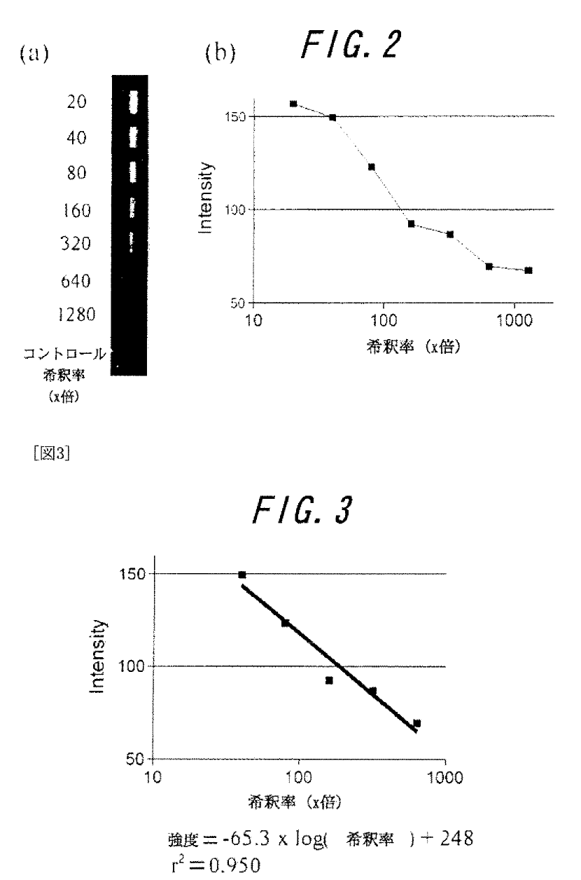

FIG. 2 shows the results of measurement of fluorescent intensities. FIG. 2 (a) shows a photograph of the fluorescent image, and the control indicates flow channels without flowing anti-mycoplasma antibody. Considering from the result that very few fluorescence was observed in the control, non-specific binding was not observed. FIG. 2 (b) shows the results of quantification of the fluorescent intensities on each spots determined from the photograph. In the result of FIG. 2 (b), when the ratio of dilution is in the range of 1/40 to 1/640, correlation was admitted between the logarithmic number of the dilution ratio and the fluorescence intensities. Meanwhile, FIG. 3 is a graph showing the correlation between the ratio of dilution of the antibody and the fluorescent intensities. When the ratio of dilution was in the range of 1/40 to 1/640, the correlation coefficient R² showed a high value of 0.950, which indicated a high correlation between them.

### Example 2

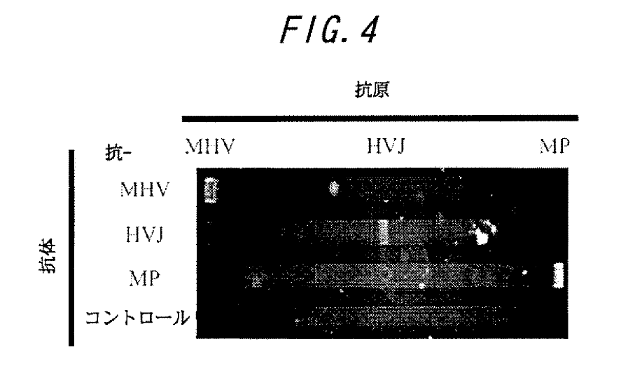

Cross reactivity was determined to examine on the cross contamination. In the same manner as example 1, antigens of pneumonia virus of Mice (MHV) (DENKA SEIKEN Co., Ltd.), Sendai virus (HVJ), and mycoplasma (MP) were sprayed by an electrospray deposition device. Thereafter anti-pneumonia virus of Mice (MHV) antibody, anti-Sendai virus (HVJ) antibody, and anti-mycoplasma (MP) antibody (DENKA SEIKEN Co., Ltd.) derived from mouse were passed through each flow channels as the primary antibodies, and the antigen antibody reactions were conducted. Then the amounts of antigens bound on the substrate were detected using anti-mouse antibody labeled with Alexa Fluor 488 as the secondary antibody. At the same time, a flow channel without flowing a primary antibody was set as a control. The results are shown in FIG. 4. According to the results, the non-specific binding of the secondary antibody was not observed in the control. On the other hand, it was revealed that respective antibodies specifically recognized the corresponding antigens.

### Example 3

Experiments on the actual samples were conducted using serum collected from mouse, The micro flow channel chip was sprayed and immobilized with the antigens of pneumonia virus of Mice (MHV) (DENKA SEIKEN Co., Ltd.), Sendai virus (HVJ), and mycoplasma (MP) using an electrospray deposition device. The test sample to be subjected to microorganism monitoring was diluted tenfold and 10 μl of the diluted sample was passed through the flow channels, then a labeled anti-mouse antibody was subjected for detection. As the result, antibody against pneumonia virus of Mice was detected in the test sample. Therefore it is assumed that the mouse is infected or has an infected record by pneumonia virus of Mice.

### INDUSTRIAL APPLICABILITY

According to this invention, an microorganism that causes infectious disease of a laboratory animal can be monitored, using a micro flow channel chip immobilized with a molecular to be tested such as an antigen or an antibody of the microorganism that causes infectious disease of the laboratory animal, by flowing serum or body fluid taken from the laboratory animal through the minute flow channel of the chip, and detecting the antigen antibody reaction on the chip. The method of the invention is useful at the place of animal experimentation, which leads to improvement in the quantity and quality of the animal experimentation. Moreover, it is assumed that this invention contributes to the development of medicines and cosmetics where animal experimentation is essential.

1. A method to monitor a microorganism that causes infectious disease of a laboratory animal, which comprises immobilizing an antigen or an antibody of a microorganism that causes infectious disease of a laboratory animal onto a micro flow channel chip directly or indirectly, flowing a test sample from the laboratory animal through micro flow channel of the micro flow channel chip, conducting an antigen antibody reaction on the micro flow channel chip, and further detecting the antigen antibody reaction.

2. The method according to claim 1, wherein said antigen or said antibody is directly or indirectly immobilized on the micro flow channel chip by electrospray deposition method.

3. The method according to claim 1, wherein said laboratory animal is mouse or rat.

4. The method according to claim 1, wherein said laboratory animal is mouse and said antigen is an antigen of a microorganism that causes infectious diseases selected from the group consisting of, Mouse hepatitis virus (MHV), Sendai virus (HVJ), Ectomrlia virus, Mouse adenovirus, Lymphocytic choriomeningitis virus (LCMV), Hantaan virus, Mycoplasma pulmonis, Clostridium piliforme, Pneumonia virus of mice, Mouse rotavirus (EDIMV), Mouse parvovirus (MVM/MPV), Mouse encephalomyelitis virus (TMEV), Pneumonia virus of Mice (PVM), Mouse Adenovirus, Reovirus type 3, Lactose dehydrogenase elevating virus, Clostridium piliforme, Corynebacterium kutscheri, Pasteurella pneumotropica, Cilia-associated respiratory (CAR) bacillus, Escherichia coli O115 a,c;K(B), Helicobactor hepaticus, Psudomonas aeruginosa, Staphylococcus aureus, Pneumocystis carinii, Giardia muris, Spironucleus muris and Helminths (pinworms).

5. The method according to claim 1, wherein said laboratory animal is rat and said antigen is an antigen of a microorganism that causes infectious disease selected from the group consisting of; Mouse hepatitis virus (MHV), Sendai virus (HVJ), Mouse adenovirus, Hantaan virus, Mycoplasma pulmonis, Clostridium piliforme, Pneumonia virus of Mice, Rat parvovirus (KRV/H-1/RPV), Mouse encephalomyelitis virus (TMEV), Pneumonia virus of Mice (PVM), Mouse Adenovirus, Reovirus type 3, Clostridium piliforme, Corynebacterium kutscheri, Bordetella bronchiseptia, Pasteurella pneumotropica, Streptococcus pneumoniae, Cilia-associated respiratory (CAR) bacillus, Psudomonas aeruginosa, Staphylococcus aureus, Pneumocystis carinii, Giardia muris, Spironucleus muris and Helminths (pinworms).

6. A method to use a micro flow channel chip, on which an antigen or an antibody of a microorganism that causes infectious disease of a laboratory animal is directly or indirectly immobilized, to monitor the microorganism.

7. The method according to claim 6, wherein said antigen or said antibody is directly or indirectly immobilized on the micro flow channel chip by electrospray deposition method.

8. The method according claim 6, wherein said laboratory animal is mouse or rat.

9. The method according to claim 6, wherein said laboratory animal is mouse and said antigen is an antigen of a microorganism that causes infectious diseases selected from the group consisting of; Mouse hepatitis virus (MHV), Sendai virus (HVJ), Ectomrlia virus, Mouse adenovirus, Lymphocytic choriomeningitis virus (LCMV), Hantaan virus, Mycoplasma pulmonis, Clostridium piliforme, Pneumonia virus of mice, Mouse rotavirus (EDIMV), Mouse parvovirus (MVM/MPV), Mouse encephalomyelitis virus (TMEV), Pneumonia virus of Mice (PVM), Mouse Adenovirus, Reovirus type 3, Lactose dehydrogenase elevating virus, Clostridium piliforme, Corynebacterium kutscheri, Pasteurella pneumotropica, Cilia-associated respiratory (CAR) bacillus, Escherichia coli O115 a,c;K(B), Helicobactor hepaticus, Psudomonas aeruginosa, Staphylococcus aureus, Pneumocystis carinii, Giardia muris, Spironucleus muris and Helminths (pinworms).

10. The method according to claim 6, wherein said laboratory animal is rat and said antigen is an antigen of a microorganism that causes infectious disease selected from the group consisting of; Mouse hepatitis virus (MHV), Sendai virus (HVJ), Mouse adenovirus, Hantaan virus, Mycoplasma pulmonis, Clostridium piliforme, Pneumonia virus of Mice, Rat parvovirus (KRV/H-1/RPV), Mouse encephalomyelitis virus (TMEV), Pneumonia virus of Mice (PVM), Mouse Adenovirus, Reovirus type 3, Clostridium piliforme, Corynebacterium kutscheri, Bordetella bronchiseptia, Pasteurella pneumotropica, Streptococcus pneumoniae, Cilia-associated respiratory (CAR) bacillus, Psudomonas aeruginosa, Staphylococcus aureus, Pneumocystis carinii, Giardia muris, Spironucleus muris and Helminths (pinworms).

11. A micro flow channel chip on which an antigen or an antibody of a microorganism that causes infectious diseases of a laboratory animal is directly or indirectly immobilized, and used to monitor the microorganism.

12. The micro flow channel chip according to claim 11, wherein said antigen or said antibody is directly or indirectly immobilized by electrospray deposition method.

13. The micro flow channel chip according to claim 11, wherein said laboratory animal is mouse or rat.

14. The micro flow channel chip according to claim 11, wherein said laboratory animal is mouse and said antigen is an antigen of a microorganism that cause infectious disease selected from the group consisting of; Mouse hepatitis virus (MHV), Sendai virus (HVJ), Ectomrlia virus, Mouse adenovirus, Lymphocytic choriomeningitis virus (LCMV), Hantaan virus, Mycoplasma pulmonis, Clostridium piliforme, Pneumonia virus of mice, Mouse rotavirus (EDIMV), Mouse parvovirus (MVM/MPV), Mouse encephalomyelitis virus (TMEV), Pneumonia virus of Mice (PVM), Mouse Adenovirus, Reovirus type 3, Lactose dehydrogenase elevating virus, Clostridium piliforme, Corynebacterium kutscheri, Pasteurella pneumotropica, Cilia-associated respiratory (CAR) bacillus, Escherichia coli O115 a,c;K(B), Helicobactor hepaticus, Psudomonas aeruginosa, Staphylococcus aureus, Pneumocystis carinii, Giardia muris, Spironucleus muris and Helminths (pinworms).

15. The micro flow channel chip according to claim 11, wherein said laboratory animal is rat and said antigen is an antigen of a microorganism that causes infectious disease selected from the group consisting of; Mouse hepatitis virus (MHV), Sendai virus (HVJ), Mouse adenovirus, Hantaan virus, Mycoplasma pulmonis, Clostridium piliforme, Pneumonia virus of Mice, Rat parvovirus (KRV/H-1/RPV), Mouse encephalomyelitis virus (TMEV), Pneumonia virus of Mice (PVM), Mouse Adenovirus, Reovirus type 3, Clostridium piliforme, Corynebacterium kutscheri, Bordetella bronchiseptia, Pasteurella pneumotropica, Streptococcus pneumoniae, Cilia-associated respiratory (CAR) bacillus, Psudomonas aeruginosa, Staphylococcus aureus, Pneumocystis carinii, Giardia muris, Spironucleus muris and Helminths (pinworms).
